(19) United States
(12) Patent Application Publication
(10) Pub. No.: US 2013/0090393 A1
(43) Pub. Date: Apr. 11, 2013

(54) PROCESS FOR PRODUCING HYDROCARBONS FROM SYNGAS

(71) Applicants: Maarten BRACHT, Amsterdam (NL); Lopamudra DEVI, Amsterdam (NL); Willem Pieter LEENHOUTS, Amsterdam (NL)

(72) Inventors: Maarten BRACHT, Amsterdam (NL); Lopamudra DEVI, Amsterdam (NL); Willem Pieter LEENHOUTS, Amsterdam (NL)

(73) Assignee: SHELL OIL COMPANY, Houston, TX (US)

(21) Appl. No.: 13/567,701

(22) Filed: Aug. 6, 2012

(30) Foreign Application Priority Data
Aug. 4, 2011 (EP) EP11176513.7

(51) Int. Cl. C07C 1/04

(52) U.S. Cl. CPC C07C 1/0485
USPC 518/702

(57) ABSTRACT

The invention pertains to a lean process for the production of hydrocarbons in which a tail gas is subjected to shift conversion, carbon dioxide removal, and then to purification in a PSA to obtain a hydrogen stream comprising more than 99 vol % hydrogen. The Fischer Tropsch process is a single-stage process. The hydrogen stream can be used to upgrade the Fischer-Tropsch hydrocarbons.

This patent application claims the benefit of European Patent Application 11176513.7 filed Aug. 4, 2011, which is incorporated herein by reference.

### FIELD OF THE INVENTION

The present invention relates to a process for the production of hydrocarbon products from syngas. Syngas is a gaseous mixture comprising hydrogen and carbon monoxide. The invention especially relates to a process in which a single Fischer-Tropsch reactor is used, and in which optimal use is made of the hydrogen comprising streams.

### BACKGROUND OF THE INVENTION

The manufacture and further processing of syngas has been found to be an attractive manner for processing various types of hydrocarbonaceous feedstock.

Sources for the manufacture of syngas are light hydrocarbon feeds, especially methane from natural sources, for example natural gas, associated gas and/or coal bed methane. There is not always the option to use the gas at its source. Transportation of gas, for example through a pipeline or in the form of liquefied natural gas, requires extremely high capital expenditure or is simply not practical. This holds true even more in the case of relatively small gas production rates and/or fields. Re-injection of gas will add to the costs of oil production, and may in the case of associated gas result in undesired effects on crude oil production. Burning of associated gas has become an undesirable option in view of depletion of hydrocarbon sources and air pollution. One of the ways to process this gas is the conversion into syngas. The gas may, for example, be converted by a gasification process such as the Shell Gasification Process. Further sources for the manufacture of syngas are the very heavy hydrocarbon fractions, or feedstock which is difficult to process by other means. Examples of this type of feedstock include peat, biomass, or coal. These materials can also be converted to syngas by gasification. After the gasification of these materials often an acid gas removal step is required in which COS, H₂5 and CO₂ are removed in order to obtain clean syngas.

The syngas manufactured from the above, or other, sources, can be converted in one or more steps over a suitable catalyst at elevated temperature and pressure into mainly paraffinic compounds ranging from methane to high molecular weight molecules comprising up to 200 carbon atoms, or, under particular circumstances, even more.

While the products obtained in a Fischer-Tropsch synthesis have attractive properties, for example low levels of contaminant like sulphur and nitrogen, they generally have a too high melting point to be directly suitable for general use as liquid fuels or lubricants. Therefore, especially the higher-boiling fractions are generally subjected to an upgrading step. The upgrading is generally performed in one or more work-up units. The upgrading step is intended to effect one or more of a decrease in viscosity, a decrease in pour point or cloud point, and a decrease in (end) boiling point.

In the art, products obtained from a Fischer-Tropsch process are often subjected to a hydrocracking step, or a hydrocracking/hydroisomerisation step.

The hydrogen required for the hydrocracking step, or the hydrocracking/hydroisomerisation step, may be obtained as follows. A hydrogen-rich syngas is made; this may be made using a furnace and/or boiler, e.g., a steam-methane reformer furnace (SMR), or a high-pressure steam boiler combined with a superheater, a gasification process and/or a gas heated reformer. One possible feed for the production of hydrogen-rich syngas is natural gas. The obtained hydrogen-rich syngas is in a next step subjected to shift conversion over a shift catalyst. During the shift conversion carbon monoxide reacts with steam to produce carbon dioxide and additional hydrogen. After the shift reaction the gas is purified in a pressure swing adsorption unit (PSA). Using the PSA, CO₂ and H₂O can be removed. The product of the PSA can comprise more than 99 vol % hydrogen, or even more than 99.9 vol % hydrogen.

The hydrocracking or hydrocracking/hydroisomerisation of a Fischer-Tropsch product is often followed by a fractionation step. The fractionation may be performed by distillation, for example using a synthetic crude distiller. Distillation fractions may be, for example, LPG, naphtha, kerosene, gas oil, and a bottom product stream. One or more boiling point fractions, especially a bottom product stream, of the hydrocracked product, or the hydrocracked/hydroisomerised product, can be subjected to a dewaxing step. A very light fraction, lighter than LPG, may be treated as off-gas and may be sent to a fuel pool.

U.S. Pat. No. 6,627,666 B1 relates to a process in which waste gas, especially acetylene off-gas, is used for a Fischer-Tropsch reaction. In FIG. 1 an overall flow diagram shows that Fischer-Tropsch tail gas (37) may be used for hydrogen recovery (34) and it may be recycled to the Fischer-Tropsch reactor (26) via compressor (36) and further processing (38) including steam reforming. The hydrogen recovery (34) is not elaborated upon in U.S. Pat. No. 6,627,666 B1. In FIG. 2 of U.S. Pat. No. 6,627,666 B1 a line-up is presented in which Fischer-Tropsch tail gas is recycled. The unsaturated hydrocarbons contained in the Fischer-Tropsch tail gas are hydrogenated (40) and excess hydrogen is stripped off (42). The gas stream is then mixed with recycled CO₂ and steam and subjected to steam reforming in an SMR (46). After removal of water and of CO₂ the purified syngas (53) is sent back to a Fischer-Tropsch reactor (26). The excess hydrogen which is stripped off (42) from the Fischer-Tropsch tail gas after the hydrogenation step may be used for hydrotreating of the Fischer-Tropsch products or as fuel for the SMR.

US20090234031A1 describes a line-up with multiple Fischer-Tropsch reactors. Exit gas from each Fischer-Tropsch reactor may partially be recycled over the same Fischer-Tropsch reactor. Additionally, exit gas from one Fischer-Tropsch reactor together with syngas with a high H₂/CO ratio is fed into a next Fischer-Tropsch reactor. The syngas with a high H₂/CO ratio preferably is prepared using a steam-methane reformer furnace (SMR).

U.S. Pat. No. 7,863,341 B2 describes a process for the preparation of syngas from two hydrocarbonaceous sources. The first source may comprise coal; the second source may comprise coal bed methane. FIG. 1 of U.S. Pat. No. 7,863,341 B2 is a diagrammatic view of a Fischer-Tropsch plant. Syngas prepared using coal gasification (10) together with syngas prepared using a steam methane reformer (SMR) (20) is lead to a

Fischer-Tropsch reactor (16). A part of the syngas produced in the SMR is sent to a high temperature shift unit (22) and then to a PSA (18), after which the produced hydrogen is used in the product work-up unit (19). The heat required for the SMR is provided by a furnace which may, for example, be powered by off-gas from the Fischer-Tropsch reactor (16).

U.S. Pat. No. 7,300,642 B1 describes an integrated plant in which a part of the generated syngas is used for a Fischer Tropsch process, and in which another part of the generated syngas is combined with Fischer Tropsch off-gas and then used to synthesize ammonia.

There is need for improvement of the process, and the present invention provides such an improved process.

The process units of the present invention are each individually known. However, joining of these process units as taught herein provides a highly advantageous process that has heretofore been unforeseen.

### SUMMARY OF THE INVENTION

The present invention provides a process for the production of hydrocarbons, which comprises the steps of:

(a) generating syngas, preferably generating syngas with an H₂/CO ratio between 1.5 and 2.3, preferably followed by removing water from the syngas;

(b) feeding at least 90 volume percent, preferably at least 95 volume %, more preferably at least 99 volume percent, of the syngas of step (a) to one Fischer-Tropsch reactor, or to two or more Fischer-Tropsch reactors which are placed in parallel;

(c) catalytic conversion of synthesis gas using a Fischer-Tropsch catalyst into a Fischer-Tropsch product in a single stage Fischer-Tropsch process;

(d) withdrawing an effluent from the Fischer-Tropsch reactor through an outlet;

(e) subjecting the effluent obtained in step (d) to a separation and/or fractionation step to form at least a heavy fraction and a light fraction which comprises unconverted syngas;

(f) optionally providing at least a part of the light fraction(s) obtained in step (e) to a scrubber and withdrawing a C₃+ hydrocarbons fraction;

(g) subjecting at least a part of the optionally scrubbed light fraction(s) to shift conversion over a shift catalyst;

(h) subjecting the shifted gas obtained in step (g) to a carbon dioxide removal;

(i) purifying the carbon dioxide lean gas obtained in step (h) in a pressure swing adsorption unit until a hydrogen stream comprising more than 99 vol % hydrogen is obtained;

(j) subjecting at least a part of the heavy fraction(s) obtained in step (e) to upgrading, preferably to hydrocracking and/or hydrocracking/hydroisomerisation, using at least a part of the hydrogen stream obtained in step (i);

wherein the process does not comprise a step in which a furnace and/or boiler is used to produce hydrogen for step (j).

### DRAWINGS

FIG. 1 shows an overview of the process steps of the present invention which is a lean method for the production of hydrocarbons.

### DETAILED DESCRIPTION OF THE INVENTION

The present invention provides a process for the production of hydrocarbons, which comprises the steps of:

(a) generating syngas, preferably generating syngas with an H₂/CO ratio between 1.5 and 2.3, preferably followed by removing water from the syngas;

(b) feeding at least 90 volume percent, preferably at least 95 volume %, more preferably at least 99 volume percent, of the syngas of step (a) to one Fischer-Tropsch reactor, or to two or more Fischer-Tropsch reactors which are placed in parallel;

(c) catalytic conversion of synthesis gas using a Fischer-Tropsch catalyst into a Fischer-Tropsch product in a single stage Fischer-Tropsch process;

(d) withdrawing an effluent from the Fischer-Tropsch reactor through an outlet;

(e) subjecting the effluent obtained in step (d) to a separation and/or fractionation step to form at least a heavy fraction and a light fraction which comprises unconverted syngas;

(f) optionally providing at least a part of the light fraction(s) obtained in step (e) to a scrubber and withdrawing a C₃+ hydrocarbons fraction;

(g) subjecting at least a part of the optionally scrubbed light fraction(s) to shift conversion over a shift catalyst;

(h) subjecting the shifted gas obtained in step (g) to a carbon dioxide removal;

(i) purifying the carbon dioxide lean gas obtained in step (h) in a pressure swing adsorption unit until a hydrogen stream comprising more than 99 vol % hydrogen is obtained;

(j) subjecting at least a part of the heavy fraction(s) obtained in step (e) to upgrading, preferably to hydrocracking and/or hydrocracking/hydroisomerisation, using at least a part of the hydrogen stream obtained in step (i);

wherein the process does not comprise a step in which a furnace and/or boiler is used to produce hydrogen for step (j).

In one embodiment, step (f) is performed and at least a part of the C₃+ hydrocarbons fraction obtained in step (f) are subjected to upgrading, preferably to hydrocracking and/or hydrocracking/hydroisomerisation, using at least a part of the hydrogen stream obtained in step (i).

In another embodiment, step (f) is performed and at least a part of the heavy fraction(s) obtained in step (e) and at least a part of the C₃+ hydrocarbons fraction obtained in step (f) are combined and subsequently subjected to upgrading, preferably to hydrocracking and/or hydrocracking/hydroisomerisation, in step (j), using at least a part of the hydrogen stream obtained in step (i).

In a process according to the invention, the process steps (a) to (i) are succeeding steps. Further steps may be performed in between or after the process steps (a) to (i) without diverting from the invention as long as the process does not comprise a step in which a furnace and/or boiler is used to produce hydrogen for step (j).

The process of the present invention is highly advantageous because it is a very lean process. It requires less equipment than known processes. For example, the process of the present invention is performed without using a furnace and/or boiler to produce hydrogen for step (j).

The carbon dioxide lean gas obtained in step (h) is not sent to a furnace and/or boiler to produce hydrogen for step (j). There also is no additional water gas shift unit required.

Especially when the catalytic conversion of step (c) is performed by providing a pressure in the range from 20 to 80 bar absolute, preferably from 30 to 70 bar absolute in the reactor, the process does not require a step in which an optionally scrubbed light fraction is subjected to compression before being subjected to the shift conversion of step (g).

Especially when the catalytic conversion of step (c) is performed by providing a pressure in the range from 20 to 80 bar absolute, preferably from 30 to 70 bar absolute in the reactor, the process does not require a step in which the carbon dioxide lean gas obtained in step (h) is subjected to compression before being purified in the pressure swing adsorption unit of step (i).

A further advantage is that the process of the invention is a simple process while the overall energy efficiency is remained high. The overall energy efficiency can be made, for example, comparable to the overall efficiency of an optimized two-stage Fischer Tropsch line-up. The thermal efficiency of the process of the present invention can be 50% or more, even 60% or more.

Yet another advantage is that the process of the invention has a relatively small carbon dioxide emission.

Another advantage is that optimal use is made of the syngas which is generated, and optionally purified, in step (a). At least 90 volume percent, preferably at least 95 volume %, more preferably at least 99 volume percent, of the syngas of step (a) is used for the Fischer-Tropsch reaction. The syngas of step (a) is not used to produce hydrogen or ammonia. The syngas of step (a) is not used as fuel. The carbon efficiency of the process of the present invention is very high. The carbon efficiency of the process of the present invention can be 60% or more, even 70% or more.

In the process of the present invention the syngas is used to a high degree in the Fischer-Tropsch reaction. The contraction in the Fischer-Tropsch reaction may be between 60 and 85%, preferably between 65 and 80%. This implies that between 60 and 85%, preferably between 65 and 80%, of the syngas is converted to hydrocarbons.

Another advantage of a process according to the present invention is that the Fischer-Tropsch off-gas produced by a single-stage Fischer-Tropsch reactor contains more hydrogen than required for the product work-up, e.g. for hydrocracking or hydrocracking/hydroisomerisation, of the hydrocarbon product of the single-stage Fischer-Tropsch reactor.

A part of the Fischer-Tropsch off-gas (HOG) can thus be used for other purposes. For example, it may be used as fuel. As the Fischer-Tropsch line-up itself has sufficient power, a part of the energy rich Fischer-Tropsch off-gas may be sold.

Alternatively, a part of the Fischer-Tropsch off-gas, which comprises a relatively large amount of hydrogen and of carbon monoxide, may be used in a chemical plant that requires syngas.

Additionally or alternatively, a part of the hydrogen obtained from the Fischer-Tropsch off-gas can be used for other purposes in addition to the product work-up of the hydrocarbon product of the single-stage Fischer-Tropsch reactor.

In step (a) syngas may be generated by gasification of a (gaseous) hydrocarbonaceous feed. The (gaseous) hydrocarbonaceous feed comprises natural gas, coal and/or biomass. Methods to convert (gaseous) hydrocarbonaceous feed into syngas include adiabatic oxidative reforming, autothermal reforming, partial oxidation, steam reforming of natural gas or liquid hydrocarbons, and gasification of coal and/or biomass. Preferably, hydrocarbonaceous feed is converted to syngas by partial oxidation at elevated temperature and pressure using an oxygen containing gas.

Partial oxidation can take place according to various established processes. Catalytic as well as non-catalytic processes may be used. These processes include the Shell Gasification Process. A comprehensive survey of this process can be found in the Oil and Gas Journal, Sep. 6, 1971, pp 86-90.

In step (a), a syngas production unit is used for the generation of syngas. Such a syngas production unit may for example be a gasification unit such as a natural gas gasification unit, a Shell Gasification Process unit, a coal gasification unit, or a biomass gasification unit.

The H₂/CO ratio of the syngas generated in step (a) preferably is between 1.5 and 2.3, more preferably between 1.8 and 2.1. After the generation of the syngas, water preferably is removed from the syngas. Also carbon dioxide, hydrogen sulfide, and other contaminants may preferably be removed from the syngas.

In step (b) at least 90 volume percent, preferably at least 95 volume %, more preferably at least 99 volume percent, of the optionally purified syngas of step (a) is fed to one or more Fischer-Tropsch reactors. If more than one Fischer-Tropsch reactor is used in step (b), the reactors are placed in parallel. Hence, a Fischer-Tropsch reactor of step (b) is not placed in series with another Fischer-Tropsch reactor in the same line-up. In the process of the present invention the Fischer Tropsch reaction of step (c) is a single stage Fischer-Tropsch process.

In step (c) synthesis gas is subjected to catalytic conversion using a Fischer-Tropsch catalyst. The syngas is converted into a Fischer-Tropsch product. Catalytic conversion in a Fischer-Tropsch reactor to which syngas is fed preferably is performed by providing the following process conditions in the reactor: a temperature in the range from 125 to 350° C., a pressure in the range from 5 to 150 bar absolute, preferably from 20 to 80 bar absolute, more preferably from 30 to 70 bar absolute, and a gaseous hourly space velocity in the range from 500 to 10000 Nl/l/h.

A part of the off-gas, or tail gas, from the Fischer-Tropsch reactor may be recycled over the same Fischer-Tropsch reactor. Hence, in a preferred embodiment at least a part of the light fraction(s) obtained in step (e) is recycled to step (b) and is fed to the Fischer Tropsch reactor together with the syngas of step (a). If desired, a hydrogen comprising stream may be fed to the Fischer Tropsch reactor together with the syngas of step (a) and at least a part of the light fraction(s) obtained in step (e). The hydrogen comprising stream may be a part of the hydrogen stream obtained in step (i).

At a hydrocarbon production site the process of the present invention may be performed using several Fischer-Tropsch reactors that are operating in parallel. In that case the same syngas preferably is supplied to more than one reactor. Additionally or alternatively, the heavy fraction(s) obtained from the effluent of more than one reactor may be combined after step (e). Additionally or alternatively, the light fraction(s) obtained from the effluent of more than one reactor may be combined after step (e).

In the present specification, the unconverted syngas comprising light fraction obtained in step (e) is also referred to as Fischer-Tropsch off-gas, hydrocarbon synthesis off-gas (HOG), heavy paraffin synthesis off-gas (HOG), and tail gas. In the present specification, the scrubbed unconverted syngas comprising light fraction obtained in step (f) is also referred to as Fischer-Tropsch off-gas, hydrocarbon synthesis off-gas (HOG), heavy paraffin synthesis off-gas (HOG), and tail gas.

In step (e) of the process of the present invention, the effluent obtained in step (d) is subjected to a separation and/or fractionation step to form at least a heavy fraction and a light fraction which comprises unconverted syngas. This may, for example, be performed by separating the Fischer-Tropsch hydrocarbon product stream from the Fischer-Tropsch off-gas by a gas/liquid separator, or by distillation.

The light fraction(s) obtained in step (e) may comprise gaseous hydrocarbons, nitrogen, unconverted methane, unconverted carbon monoxide, carbon dioxide, hydrogen and water. The gaseous hydrocarbons are suitably C₁-C₅ hydrocarbons, preferably C₁-C₄ hydrocarbons, more preferably C₁-C₃ hydrocarbons. These hydrocarbons, or mixtures thereof, are gaseous at temperatures of 5-30° C. (1 bar), especially at 20° C. (1 bar). Further, oxygenated compounds, e.g. methanol, dimethylether, may be present. The hydrogen content in the light fraction obtained in step (e) preferably is at least 10 volume %, more preferably at least 15 volume %. The hydrogen content in the light fraction obtained in step (e) may be less than 40 volume %, even less than 30 volume %.

In optional step (f) at least a part of the light fraction(s) obtained in step (e) are provided to a scrubber and a C₃+ hydrocarbons fraction is withdrawn. The scrubber used in step (f) may be a wet scrubber. Preferably the Fischer Tropsch off-gas, or tail gas, is contacted with a wash fluid in a scrubber. When entering the scrubber, the Fischer Tropsch off-gas preferably is at a temperature of 0 to 50° C., more preferably 10 to 40° C. When entering the scrubber, pressure of the Fischer Tropsch off-gas preferably is 1-80 bar, more preferably 20-70 bar.

Preferably the scrubber is adapted to provide maximum contact between the Fischer-Tropsch tail gas and the wash fluid with minimum pressure drop. Preferably the pressure during the contacting step is the same as the Fischer-Tropsch tail gas pressure. The wash fluid typically comprises hydrocarbons. Preferably the wash fluid comprises C₅-C₂₀ hydrocarbons, more preferably C₈-C₂₀, even more preferably C₈-C₁₄ or C₁₀-C₁₄. Preferably the initial boiling point of the wash fluid is higher than 80° C., more preferably higher than 100° C. The higher the initial boiling point of the wash fluid the easier it is to separate the Fischer-Tropsch tail gas component, i.e. the C₃+ hydrocarbons fraction, from the wash fluid. The wash fluid is preferably kerosene or gasoil. Preferably the wash fluid is not naphtha.

The wash fluid may be circulated through the scrubber more than once. Preferably a bleed stream is provided to remove a portion of the wash fluid on each cycle. Preferably a make-up stream is provided to add a portion of the wash fluid on each cycle. Typically the Fischer-Tropsch tail gas passes through the scrubber only once.

Preferably at least 70 wt %, more preferably at least 90 wt % of the C₃-C₉ hydrocarbons in the Fischer-Tropsch tail gas stream are removed. Preferably at least 70 wt %, more preferably at least 90 wt % of the C₄-C₇ hydrocarbons in the Fischer-Tropsch tail gas stream are removed. The amount of hydrocarbons removed from the Fischer-Tropsch tail gas stream may, for example, be increased by lowering the temperature in the scrubber or by increasing amount of wash fluid with respect to the Fischer-Tropsch tail gas stream.

A separation unit, such as a distillation unit, is preferably provided to separate the wash fluid from the Fischer-Tropsch tail gas component. Strippers, flashers or any other suitable separation units may also be used. If the optional scrubbing of step (f) is performed, at least a part of the remaining Fischer-Tropsch tail gas is subjected to shift conversion over a shift catalyst in step (g).

At least a part of the optionally scrubbed light fraction(s) obtained in step (e) is subjected to shift conversion over a shift catalyst in step (g). The shift catalyst is sometimes referred to as a water-gas-shift (WGS) catalyst. During the shift conversion carbon monoxide reacts with steam to produce carbon dioxide and additional hydrogen. Examples of suitable shift catalysts are an iron and iron oxide catalysts, iron oxide promoted with chromium oxide, and copper on a mixed support composed of zinc oxide and aluminum oxide. The hydrogen content in the shifted gas obtained in step (g) preferably is at least 25 volume %, more preferably at least 30 volume %. The hydrogen content in the shifted gas obtained in step (g) may be less than 60 volume %, even less than 50 volume %.

In step (h) the shifted gas obtained in step (g) is subjected to a carbon dioxide removal. A carbon dioxide rich stream and a carbon dioxide lean gas are obtained. The shifted gas that is subjected to carbon dioxide removal may be at a temperature in the range of 0-100° C., and at a pressure in the range of 1-80 bar.

For the removal of carbon dioxide any suitable conventional process may be used, for instance adsorption processes using amines, especially in combination with a physical solvent, such as the ADIP process or the SULFINOL process as described in inter alia GB 1,131,989; GB 965,358; GB 957260; and GB 972,140, or the ADIP-X process described in inter alia the contribution of J. B. Rajani to the 12^{th} congress of the Research Institute of Petroleum Industry (RIPI) “Treating Technologies of Shell Global Solutions for Natural Gas and Refinery Gas Streams” of 2004. Carbon dioxide removal is often referred to as carbon capture, which is usually part of carbon capture and storage processes.

The carbon dioxide rich stream obtained in step (h) may be stored or re-used. CO₂ storage may for example, include gaseous storage in various deep geological formations (including saline formations and exhausted gas fields), liquid storage in the ocean, and/or solid storage by reaction of CO₂ with metal oxides to produce stable carbonates. Carbon dioxide storage is often referred to as CO₂ sequestration, which is usually part of carbon capture and storage processes (CCS). Additionally or alternatively, CO₂ may be re-used for enhanced oil recovery and/or for plant growth and production within a greenhouse environment and/or for pelleting and using in industrial cooling applications.

In step (h) preferably at least 70 vol. %, more preferably between 60 and 80 vol. %, even more preferably at least 90 vol. % of CO₂ is removed from the shifted gas obtained in step (g), calculated on the total amount of CO₂ in the shifted gas obtained in step (g). The hydrogen content in the carbon dioxide lean gas obtained in step (h) preferably is at least 30 volume %, more preferably at least 40 volume %. The hydrogen content in the carbon dioxide lean gas obtained in step (h) may be less than 80 volume %, even less than 75 volume %.

In step (i), the carbon dioxide lean gas obtained in step (h) is purified in a pressure swing adsorption unit (PSA) until a hydrogen stream comprising more than 99 vol % hydrogen is obtained. Preferably the carbon dioxide lean gas obtained in step (h) is purified in the PSA until a hydrogen stream comprising more than 99.9 vol % hydrogen is obtained.

Optionally, at least a part of the heavy fraction(s) obtained in step (e) are subjected to upgrading. Optionally, at least a part of the C₃+ hydrocarbons fraction obtained in a scrubbing step (f) are subjected to upgrading. In one embodiment, at least a part of the heavy fraction(s) obtained in step (e) and at least a part of the C₃+ hydrocarbons fraction obtained in a scrubbing step (f) are combined and then subjected to upgrading. Upgrading may be performed in one or more product work-up units. Upgrading may be performed by hydrocracking or hydrocracking/hydroisomerisation, optionally followed by distillation. In one embodiment at least a part of the hydrocarbons that are subjected to hydrocracking or hydrocracking/hydroisomerisation is hydrogenated before hydrocracking or hydrocracking/hydroisomerisation.

At least a part of the heavy fraction(s) obtained in step (e) are subjected to upgrading using at least a part of the hydrogen stream obtained in step (i). For example, hydrogenation of at least a part of the heavy fraction(s) obtained in step (e) may be performed using at least a part of the hydrogen stream obtained in step (i). Additionally or alternatively, hydrocracking or hydrocracking/hydroisomerisation of at least a part of the heavy fraction(s) obtained in step (e) may be performed using at least a part of the hydrogen stream obtained in step (i).

The process of the invention may comprise water separation steps. For example, water may be separated from the shifted gas obtained in step (g) before it is subjected to carbon dioxide removal in step (h). Additionally or alternatively, water may be separated from the carbon dioxide lean gas obtained in step (h) before it is purified in step (i). Water separation can be performed with any suitable technique, for example using a flash vessel.

The Fischer-Tropsch reactor that may be used in the present invention preferably contains a Fischer-Tropsch catalyst. Preferably the Fischer-Tropsch catalyst comprises a Group VIII metal component, more preferably cobalt, iron and/or ruthenium, most preferably cobalt. References to the Periodic Table and groups thereof used herein refer to the previous IUPAC version of the Periodic Table of Elements such as that described in the 68th Edition of the Handbook of Chemistry and Physics (CPC Press).

Typically, the catalysts comprise a catalyst carrier. The catalyst carrier is preferably porous, such as a porous inorganic refractory oxide, more preferably alumina, silica, titania, zirconia or combinations thereof, most preferably titania.

The optimum amount of catalytically active metal present on the carrier depends inter alia on the specific catalytically active metal. Typically, the amount of cobalt present in the catalyst may range from 1 to 100 parts by weight per 100 parts by weight of carrier material, preferably from 10 to 50 parts by weight per 100 parts by weight of carrier material. In case the catalyst comprises cobalt and titania, the amount of cobalt preferably is in the range of between 10 weight percent (wt %) and 35 wt % cobalt, more preferably between 15 wt % and 30 wt % cobalt, calculated on the total weight of titania and cobalt.

In step (j) at least a part of the hydrogen stream obtained in step (i) is used to upgrade at least a part of the heavy fraction(s) obtained in step (e). In step (j) preferably hydrocracking or hydrocracking/hydroisomerisation takes place.

Step (j) may be performed at another location than the location at which the Fischer-Tropsch process step (c) is performed. The process of the present invention does not comprise a step in which a furnace and/or boiler is used to produce hydrogen for step (j).

A hydrocracking or hydrocracking/hydroisomerisation catalyst may be used in step (j). In that case preferably a catalyst is used which contains a catalytically active metal component as well as an acidic function. The metal component can be deposited on any acid carrier having cracking and isomerisation activity, for example a halogenated (e.g. fluorided or chlorided) alumina or zeolitic carrier or an amorphous silica/alumina carrier.

Preferably the hydrocracking or hydrocracking/hydroisomerisation catalyst comprises a mixture of two refractory oxides, more preferably amorphous silica/alumina. Preferably the catalyst comprises a zeolite, preferably zeolite beta, in addition to amorphous silica/alumina. The silica:alumina ratio of the zeolite (beta) most preferably is in the range from 5 to 500, more preferably in the range from 50 to 300.

The catalyst used in the hydrocracking/hydroisomerisation step may contain as catalytically active metal components one or more metals selected from Groups VIB, VIIB and/or VIII of the Periodic System. Examples of such metals are molybdenum, tungsten, rhenium, the metals of the iron group and the metals of the platinum and palladium groups. Catalysts with a noble metal as catalytically active metal component generally contain 0.05-5 parts by weight and preferably 0.1-2 parts by weight of metal per 100 parts by weight of carrier material. Very suitable noble metals are palladium and platinum. Catalysts with a non-noble metal or a combination of non-noble metals as catalytically active metal component generally contain 0.1-35 parts by weight of metal or combination of metals per 100 parts by weight of carrier material. Very suitable hydrocracking catalysts contain a combination of 0.5-20 parts by weight and in particular 1-10 parts by weight of a non-noble metal of Group VIII and 1-30 parts by weight and in particular 2-20 parts by weight of a metal of Group VIB and/or VIIB per 100 parts by weight of carrier material. Particularly suitable metal combinations are combinations of nickel and/or cobalt with tungsten and/or molybdenum and/or rhenium Likewise very suitable as hydrocracking catalysts are catalysts which contain 0.1-35 parts by weight and in particular 1-15 parts by weight of nickel per 100 parts by weight of carrier material.

If the hydrocracking or hydrocracking/hydroisomerisation catalysts contain a non-noble metal or combination of non-noble metals as catalytically active metal component, they are preferably used in their sulphidic form. The conversion of the catalysts to their sulphidic form can very suitably be carried out by contacting the catalysts at a temperature below 500° C. with a mixture of hydrogen and hydrogen sulphide in a volume ratio of 5:1 to 15:1. The conversion of the catalysts into the sulphidic form may also be carried out by adding to the feed, under reaction conditions, sulphur compounds in a quantity of from 10 ppmw to 5% by weight and in particular in a quantity of from 100 ppmw to 2.5% by weight.

Most preferably the hydrocracking or hydrocracking/hydroisomerisation catalyst comprises platinum and/or palladium and an amorphous silica/alumina, and optionally zeolite beta.

The effluent from the reaction zone in which hydrocracking or hydrocracking/hydroisomerisation takes place is subjected to a fractionation step, for example a distillation. The fractionation may be performed by distillation, for example using a synthetic crude distiller. The effluent is fractionated into at least a heavy fraction and a hydrogen-rich light fraction, and preferably into at least a heavy fraction, an intermediate fraction, and a hydrogen-rich fraction.

Distillation fractions may be, for example, LPG, naphtha, kerosene, gas oil, and a bottom product stream. One or more boiling point fractions, especially a bottom product stream, of the hydrocracked product, or the hydrocracked/hydroisomerised product, can be subjected to a dewaxing step. A very light fraction, preferably lighter than LPG, is a hydrogen-rich light fraction. The hydrogen-rich light fraction may contain more than 50 vol % hydrogen, preferably more than 60 vol % hydrogen. The amount of hydrogen in the hydrogen-rich light fraction can be influenced by the partial hydrogen pressure and the temperature used in the hydrocracking, or hydrocracking/hydroisomerisation step (j). The partial hydrogen pressure used in the hydrocracking, or hydrocracking/hydroisomerisation step (j) preferably is in the range of from 20 bar to 70 bar.

One embodiment of the process according to the invention will be illustrated below with reference to the attached figure. It is noted that the present invention should not be considered limited thereto or thereby.

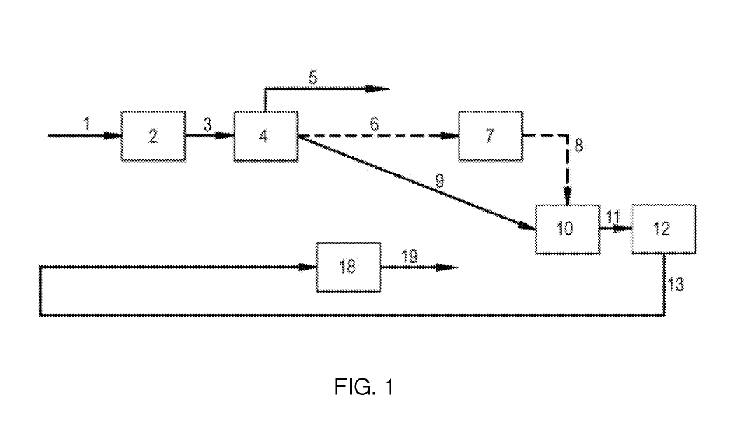

FIG. 1 illustrates a process according to the invention. Natural gas (1) is fed to a syngas production unit (2). Syngas (3) is fed to a Fischer-Tropsch reactor (4). A heavy fraction of the effluent of the Fischer Tropsch reactor (4) is fed through line (5) to a product work-up unit (not shown). A light fraction of the effluent of the Fischer-Tropsch reactor (4) can be fed via line (6) to a scrubber (7) and then to a shift unit (10). Alternatively, a light fraction of the effluent is fed via line (9) directly to a shift unit (10). Shifted gas is fed via line (11) to a CO₂ removal unit (12). Carbon dioxide lean gas is fed via line (13) fed to a PSA unit (18). Optionally water is removed before the carbon dioxide lean gas is fed to the PSA unit (18). The obtained hydrogen stream (19) can be used to upgrade the heavy fraction of the effluent of the Fischer Tropsch reactor (4) which is fed through line (5) to a product work-up unit (not shown).

In the process according to the invention as illustrated in FIG. 1, the carbon dioxide lean gas obtained in the CO₂ removal unit (12) is not sent to a furnace and/or boiler; the carbon dioxide lean gas is fed via line (13) to a PSA unit (18).

What is claimed is:

1. A process for the production of hydrocarbons, which comprises the steps of:
(a) generating syngas;
(b) feeding at least 90 volume percent, of the syngas of step (a) to one Fischer-Tropsch reactor, or to two or more Fischer-Tropsch reactors which are placed in parallel;
(c) catalytic conversion of synthesis gas using a Fischer-Tropsch catalyst into a Fischer-Tropsch product in a single stage Fischer-Tropsch process;
(d) withdrawing an effluent from the Fischer-Tropsch reactor through an outlet;
(e) subjecting the effluent obtained in step (d) to a separation and/or fractionation step to form at least a heavy fraction and a light fraction which comprises unconverted syngas;
(f) providing at least a part of the light fraction(s) obtained in step (e) to a scrubber and withdrawing a C₃+ hydrocarbons fraction;
(g) subjecting at least a part of the optionally scrubbed light fraction(s) to shift conversion over a shift catalyst;
(h) subjecting the shifted gas obtained in step (g) to a carbon dioxide removal;
(i) purifying the carbon dioxide lean gas obtained in step (h) in a pressure swing adsorption unit until a hydrogen stream comprising more than 99 vol % hydrogen is obtained;
(j) subjecting at least a part of the heavy fraction(s) obtained in step (e) to hydrocracking and/or hydrocracking/hydroisomerisation, using at least a part of the hydrogen stream obtained in step (i);
wherein the process does not comprise a step in which a furnace and/or boiler is used to produce hydrogen for step (j).

2. The process according to claim 1, wherein the catalytic conversion of step (c) is performed by providing the following process conditions in the reactor: a temperature in the range from 125 to 350° C., a pressure in the range from 5 to 150 bar absolute, and a gaseous hourly space velocity in the range from 500 to 10000 Nl/l/h.

3. The process according to claim 1, wherein at least a part of the light fraction(s) obtained in step (e) is recycled to step (b) and is fed to the Fischer Tropsch reactor together with the syngas provided in step (a).

4. The process according to claim 1, wherein at least a part of the heavy fraction(s) obtained in step (e) and at least a part of the C₃+ hydrocarbons fraction obtained in step (f) are combined and subsequently subjected to hydrocracking and/or hydrocracking/hydroisomerisation, in step (j), using at least a part of the hydrogen stream obtained in step (i).

5. The process according to claim 1, wherein the catalytic conversion of step (c) is performed by providing the following process conditions in the reactor: a temperature in the range from 125 to 350° C., a pressure in the range from 20 to 80 bar absolute, and a gaseous hourly space velocity in the range from 500 to 10000 Nl/l/h,
and wherein the process does not comprise a step in which an optionally scrubbed light fraction is subjected to compression before being subjected to the shift conversion of step (g).

6. The process according to claim 1, wherein the catalytic conversion of step (c) is performed by providing the following process conditions in the reactor: a temperature in the range from 125 to 350° C., a pressure in the range from 20 to 80 bar absolute, and a gaseous hourly space velocity in the range from 500 to 10000 Nl/l/h,
and wherein the process does not comprise a step in which carbon dioxide lean gas obtained in step (h) is subjected to compression before being purified in the pressure swing adsorption unit of step (i).

7. The process according to claim 1, wherein the hydrogen content in the light fraction obtained in step (e) is at least 10 volume %.

8. The process according to claim 1, wherein the hydrogen content in the shifted gas obtained in step (g) is at least 25 volume %.

9. The process according to claim 1, wherein the hydrogen content in the carbon dioxide lean gas obtained in step (h) is at least 30 volume %.

10. A process for the production of hydrocarbons, which comprises the steps of:
(a) generating syngas with an H₂/CO ratio between 1.5 and 2.3, followed by removing water from the syngas;
(b) feeding at least 90 volume percent, of the syngas of step (a) to one Fischer-Tropsch reactor, or to two or more Fischer-Tropsch reactors which are placed in parallel;
(c) catalytic conversion of synthesis gas using a Fischer-Tropsch catalyst into a Fischer-Tropsch product in a single stage Fischer-Tropsch process;
(d) withdrawing an effluent from the Fischer-Tropsch reactor through an outlet;
(e) subjecting the effluent obtained in step (d) to a separation and/or fractionation step to form at least a heavy fraction and a light fraction which comprises unconverted syngas;
(f) providing at least a part of the light fraction(s) obtained in step (e) to a scrubber and withdrawing a C₃+ hydrocarbons fraction;
(g) subjecting at least a part of the optionally scrubbed light fraction(s) to shift conversion over a shift catalyst;
(h) subjecting the shifted gas obtained in step (g) to a carbon dioxide removal;
(i) purifying the carbon dioxide lean gas obtained in step (h) in a pressure swing adsorption unit until a hydrogen stream comprising more than 99 vol % hydrogen is obtained;
(j) subjecting at least a part of the heavy fraction(s) obtained in step (e) to hydrocracking and/or hydrocracking/hydroisomerisation, using at least a part of the hydrogen stream obtained in step (i);
wherein the process does not comprise a step in which a furnace and/or boiler is used to produce hydrogen for step (j).

11. A process for the production of hydrocarbons, which comprises the steps of:
(a) generating syngas with an H₂/CO ratio between 1.5 and 2.3, followed by removing water from the syngas;
(b) feeding at least 90 volume percent, of the syngas of step (a) to one Fischer-Tropsch reactor, or to two or more Fischer-Tropsch reactors which are placed in parallel;
(c) catalytic conversion of synthesis gas using a Fischer-Tropsch catalyst into a Fischer-Tropsch product in a single stage Fischer-Tropsch process;
(d) withdrawing an effluent from the Fischer-Tropsch reactor through an outlet;
(e) subjecting the effluent obtained in step (d) to a separation and/or fractionation step to form at least a heavy fraction and a light fraction which comprises unconverted syngas;
(f) subjecting at least a part of the heavy fraction(s) obtained in step (e) to hydrocracking and/or hydrocracking/hydroisomerisation;
wherein the process does not comprise a step in which a furnace and/or boiler is used to produce hydrogen for step (f).
